(19) United States
(12) Patent Application Publication
(10) Pub. No.: US 2010/0137997 A1
(43) Pub. Date: Jun. 3, 2010

(54) PROCESS FOR REALISING BIOLOGICALLY-COMPATIBLE THREE-DIMENSIONAL ELEMENTS

(75) Inventors: Francesco Casari, Smarano( TN) (IT); Luca Girardini, Cimego (TN) (IT); Alberto Molinari, Mezzocorona (TN) (IT); Mario Zadra, Tres (TN) (IT)

Correspondence Address: COLLARD & ROE, P.C., 1077 NORTHERN BOULEVARD, ROSLYN, NY, 11576, US

(73) Assignee: K4SINT S.r.l.

(21) Appl. No.: 12/452,241

(22) PCT Filed: Jun. 27, 2008
(86) PCT No.: PCT/IB2008/001686

§ 371 Date: Dec. 22, 2009

(30) Foreign Application Priority Data
Jul. 4, 2007 (IT) MO2007A000223

(51) Int. Cl. A61F 2/30; B05D 3/00

(52) U.S. Cl. 623/23.43; 427/2.26

(57) ABSTRACT

The process for realizing biologically-compatible three-dimensional elements comprises sintering in forming means (6) and at a sintering temperature (T1), by passing an electrical current and applying a pressure, a volume (11) of forming material, obtaining formed concave and/or convex elements (1). Before the sintering operation, a granular material (12) is added to the volume (11) of forming material, which granular material (12) is removable from the formed concave and/or convex elements (1), having a melting temperature (T2) which is higher than the sintering temperature (T1); in this way pores (5) are obtained.

### TECHNICAL FIELD

The invention relates to a process for realising biologically-compatible three-dimensional elements, particularly for realising prostheses which can be implanted in the human and animal bodies.

### BACKGROUND ART

The prior art comprises production of concave and convex elements which are biologically compatible, such as for example, cotyls, which are used for realising prostheses that are destined to be implanted in the human or animal body in order to replace bone parts or joints which have been irredeemably damaged by trauma or disease such as arthritis and arthrosis.

The production of these concave and/or convex elements is done using different methods.

In a first method, the concave or convex element is made in its final form directly out of a solid body, by subjecting the solid body to modelling operations by means of removal of shavings, up until the desired size and shape is obtained.

A second known method includes realising concave-convex shapes by means of hot or cold plastic deformation of a solid body.

In a further method which uses metal powders, known as “Rapid Prototyping”, using technologies known as “Selective Laser Sintering” (SLS) and “Electron Beam Melting” (EBM), concave and convex elements are formed by depositing and sintering power layers, such as to obtain the elements by accumulation of successive layers, made cohesive by sintering.

In other words, in each layer zones are created which have a predetermined shape in which the powders are cohesive, and zones in which the powders remain in the loose state.

In this method, the instrument which produces the sintering is a laser emitter (SLS, Selective Laser Sintering) or an electron emitter (EBM, Electron Beam Melting) which strike the powders in the zones defined by the outlines to be realised. When the concave and convex elements are obtained by one of the previously-listed methods, a second working stage is required in order to predispose the external convex surfaces thereof such that they are easily and stably connectable to the bones of the body in which they will be implanted, without however giving rise to phenomena related to bio-incompatibility.

The predisposing of these external convex surfaces, which when the elements are first realised are substantially smooth, consists in making them porous or creating roughness thereon, such that the process of osteo-integration is accelerated and better mechanical stability of the implanted element is ensured.

Further, this porosity or roughness must be realised using suitable materials in order to facilitate natural blending of the concave and/or convex elements into the bone apparatus, such as, e.g., hydroxyapatite or titanium and its alloys.

It is stressed that these concave and convex elements, or cotyls, are used especially for realising hip-joint prostheses and that they have to be able to withstand the forces and stresses deriving from the body weight and walking.

To obtain the porosity or roughness, the gripping surfaces, generally the convex ones, of the concave and convex elements are coated with a layer of rough or porous additive substances which make the elements able to grip and be biologically compatible with the human or animal body, such as hydroxyapatite or titanium and its alloys, as mentioned above, using a process known as Plasma Spray, in which the hydroxyapatite, the titanium or other biocompatible substance is sprayed onto the gripping surfaces of the concave and convex elements such as to create the desired roughness.

Another way of obtaining this roughness is to realise it directly during the forming of the elements, using the method known as “Rapid Prototyping”.

However, in both cases, the prior art exhibits some drawbacks. A first drawback is that in the case of application by Plasma Spray the spraying energy has to be kept at a limited level to prevent the hydroxyapatite particles, or titanium and/or titanium alloy particles, or other biocompatible material being sprayed, from deforming by flattening on the application surface and creating a lower level of roughness which is inadequate for good osteo-integration.

This limitation of spraying energy leads to poor adhesion of the particles, with a subsequent detachment of the particles from the coated surface. This drawback must be absolutely avoided since the detached particles over time would be released into the organism after the prosthesis had been implanted.

The forming of the externally porous concave and convex elements, or cotyls, using the Rapid Prototyping method exhibits this very drawback of leaving non-cohesive particles which are therefore liable to be released into the organism.

Since the progressive forming of the concave elements is done in an immersion of powder layers, of which only the sintered portions are actually cohesive, non-cohesive particles can collect internally of the micro-pores created during the forming process.

A further drawback of the prior art is that some methods used for realising biologically-compatible concave and convex elements require considerably long realisation times.

Further, other methods require a combination and sequence of different technologies in order to realise the element and the porous part.

Other methods, such as mechanical working from a solid piece, exhibit the further drawback of creating large amounts of waste materials, thus adding to the production costs.

### OBJECTS OF THE INVENTION

An object of the invention is to improve the state of the prior art.

A further object of the invention is to realise a process for realising biologically-compatible concave and convex elements which are particularly suitable for realising prostheses which can be implanted in the human and animal body, which enables obtaining these concave and convex elements in a single working stage, and ready for use.

A further object of the invention is to set up a process for realising biologically-compatible concave and convex elements which enables a rapid and complete sintering of the forming materials to be made, whether the materials are electrically conductive or not.

In an aspect of the invention, a process is provided for realising biologically-compatible concave and convex elements which comprises:
sintering in forming means and at a sintering temperature a volume of forming material of an electrically conductive/non-conductive type, obtaining formed concave and convex elements;
adding to said forming material, before said sintering, a granular material which is removable from said formed concave and convex elements, said granular material (12) having a melting point temperature which is higher than said sintering temperature, characterised in that said granular material (12) comprises granular material having a low level of electrical conductivity when said forming material is of an electrically-conductive type or granular material having a high level of electrical conductivity when said forming material is of a substantially electrically non-conductive type.

The process for realising biologically-compatible concave and convex elements, particularly suitable for realising prostheses which can be implanted in the human and animal body enables obtaining these concave and convex elements in a single work stage, reducing the times and costs of production and eliminating the waste materials created thereby.

The process for realising biologically-compatible concave and convex elements further enables a rapid sintering of the forming material, whether it is electrically conductive or non-conductive.

### BRIEF DESCRIPTION OF THE DRAWINGS

Further characteristics and advantages of the invention will better emerge from the description that follows of a process for realising biologically-compatible concave and convex elements, particularly suitable for realising prostheses which can be implanted in the human and animal body, illustrated by way of non-limiting example in the accompanying figures of the drawings, in which:

FIG. 1 is a perspective view of a cotyl obtained using a process for realising biologically-compatible elements;

FIG. 2 is a very schematic view in vertical section of a die comprising a matrix, a concave punch and a convex punch for obtaining the cotyl of FIG. 1, in a configuration in which the convex punch is extracted from the matrix;

FIG. 3 is a very schematic view in vertical section of the die of FIG. 2, in a stage of formation of the cotyl of FIG. 1;

FIG. 4 is a section and interrupted view in enlarged scale of a detail of FIG. 3;

FIG. 5 is a section and interrupted view in enlarged scale of a portion of the cotyl of FIG. 1.

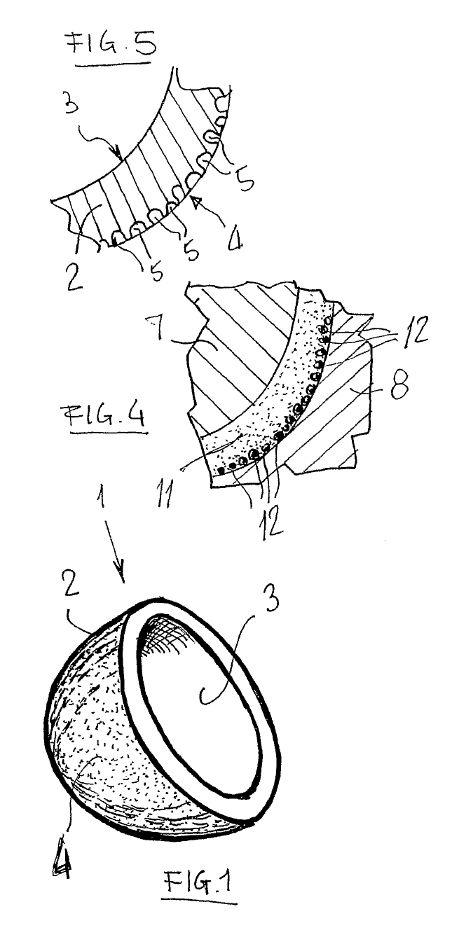

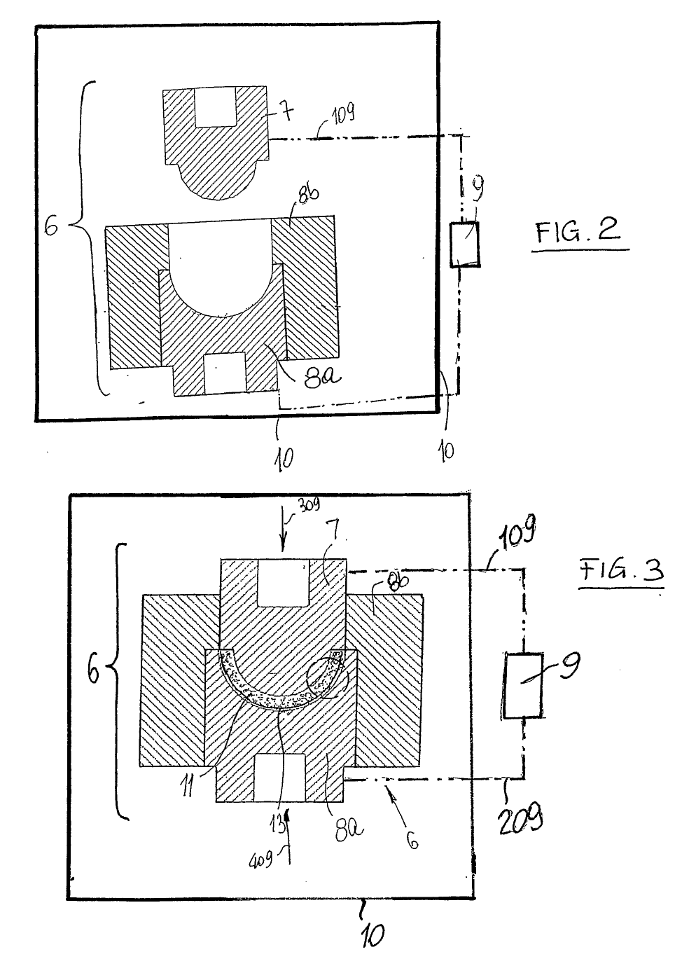

### PREFERRED EMBODIMENTS OF THE INVENTION

With reference to the figures of the drawings, 1 denotes a cotyl which can be implanted in an acetabulum of a patient's hip joint and which exhibits a cup-shaped body 2, which defines a concave surface 3 and a convex surface 4.

The convex surface 4 exhibits a plurality of cells 5 which enable the cotyl 1 to be arranged internally of the acetabulum such that it does not move with respect thereto and such that incorporation into the patient's bone is facilitated.

The cotyl 1 is normally part of a prosthesis which is applied on the patient to replace the damaged hip-joint and to enable rotatable coupling with an artificial spherical head applied at the head of a patient's femur, which completes the hip prosthesis.

The forming of the cotyl 1, which in this example is cup-shaped, but which could also exhibit other known concave shapes, familiar to an expert in the field and according to other specific requirements and applications, is realised by means of a die 6 which comprises a convex punch 7 and a concave punch 8a and a matrix 8b which is inserted in a Spark Plasma Sintering system for realising forming and sintering processes.

This Spark Plasma Sintering system comprises, e.g., a pulse current generator 9 which is connected by means of the lines 109 and 209 both with the convex punch 7 and with the concave punch 8a, realised using electrically-conductive materials such as, e.g., graphite, thus contemporaneously becoming electrodes.

The expert in the field can however choose to realise both the convex punch 7 and the concave punch 8a and the matrix 8b using different materials from graphite, such as, e.g., Aluminium oxide, zirconium oxide, Tungsten Carbide, such as to improve the distribution of the temperature according to the needs of the process.

The expert in the field can also realise both the convex punch 7 and the concave punch 8a and the matrix 8b using portions of mixed structure, i.e. partially made of graphite and partially of aluminium oxide, zirconium oxide, Tungsten Carbide.

Together with the action of the electrical current which by the Joule effect causes the heating of the convex punch 7 and the concave punch 8a and matrix 8b, a coaxial compression force is applied on the convex punch 7 and the concave punch 8a and the matrix 8b, which compression force is represented by the arrows 309 and 409, for example using a hydraulic apparatus.

The die 6 is in turn inserted internally of a vacuum chamber, denoted schematically by the broken lines 10.

A layer 11 of a powder material is arranged in the concave punch 8a, which powder material can be of an electrically-conductive type or can be electrically non-conductive and which normally comprises titanium or another biocompatible material or alloy, such as, e.g., chromium-cobalt, stellites, nickel-titanium, alumina, zirconium, hydroxyapatite, to form the cotyl 1.

The layer 11 of powder is destined to be sintered during the pressing in the die 6; however in order for the external surface of the cotyl 1 to be made porous as is desired, i.e. for concave cells 5 to be present thereon to facilitate the gripping of the cotyl 1 to the bone tissues of the patient, a quantity of granular material is added to the layer 11, the granular material containing granules 12 having selected dimensions and being later eliminated, e.g. dissolved using a solvent or via a thermo-chemical treatment.

By the term “added”, it is meant that the granular material can be mixed with the powder material or can constitute a further layer 13 arranged internally of the concave punch 8a before the layer 11 is introduced.

The granular material used is selected from among those which have a melting temperature T2 which is higher than the sintering temperature T1 of the layer of powder materials, e.g. titanium, and, for this reason, when the layer 11 is formed and sintered internally of the die 6, the granules 12 are sunk therein, maintaining their shape.

According to the invention, the granules 12 are selected to be electrically conductive or poorly conductive, according to the type of forming material, i.e. the layer of powder 11.

In other words, when the forming material is conductive, the granules 12 are non-conductive (or poorly conductive) such that the electrical sintering current is concentrated and passes only through the forming material, heating it up and sintering it.

On the contrary, when the forming material or alloy is non-conductive, or poorly conductive, the selected granules 12 are of an electrically-conductive material (for example graphite) so that the sintering current passes through the granules 12 and heats them up, and from the granules 12 the heat is also transmitted, by conduction, to the forming material and sinters it.

Satisfactory results have been obtained from the following examples, which are not intended to be limiting.

### Example 1

Commercially pure grade 1 titanium powder (cpTi grade 1, density 4.51 g/cm3), dimensions from 20 to 45 micron, is mixed with granules 12 of Sodium Chloride (NaCl, density 2.16 g/cm3), dimensions from 500 to 1000 micron, in volume ratio 35% cpTi grade 1 and 65% NaCl. The addition of acetone or another solvent can facilitate the homogenisation of the powder with the granules, such that the granules 12 are uniformly covered with powder. The concave punch 8a, the convex punch 7 and the compact isotropic graphite matrix 8b are used, with a resistance to flexion greater than 65 MPa. The concave punch 8a is inserted in the matrix 8b and filled with the mixture of powder 11 and granules 12, such that the effective volume of material introduced is equal to the final volume desired for the porous layer: for a thickness of the porous layer of 1.89 mm and an external diameter of the acetabular cup (or cotyl) 1 of 50 mm, a total effective powder 11 and granule 12 volume of 4.36 cm³ is introduced, i.e. a mass of commercially pure grade 1 titanium powder of 6.88 g and a mass of sodium chloride of 6.12 g.

The powder 11 and granule 12 mixture, possibly wetted with acetone such as to render it suitably plastic and formable, is distributed in a uniform layer on the concave surface of the concave punch 8a. This operation can be facilitated with special hemispherical convex tools. Alternatively the powder and granule 12 mixture can be previously and separately formed into a cup-shape 2 with the use of one or more different forming technologies, e.g. tape casting, injection in hollow dies, moulding, and thereafter can be loaded into the concave punch 8a, after the external forming operations. The punch 8a and the matrix 8b are loaded with a volume 11 of commercially pure grade 1 titanium powder, necessary for obtaining the acetabular cup (or cotyl) 1 having the desired shape and volume following complete densification of the powder 11 by means of Spark Plasma Sintering.

The convex punch 7 is then inserted into the matrix 8b such that the convex surface of the punch 7 goes into contact with the powder 11 to be sintered and formed in the geometry of the convex punch 7 itself. The quality of the surface finishing of the convex punch 7 will determine the degree of roughness of the internal surface of the acetabular cup 1 and will reduce the mechanical working necessary for finishing the internal surface.

The system of punches, matrix and powders is therefore placed under vacuum, at a pressure of 6 Pa, and then heated at a velocity of about 100° C./min up to a sintering temperature of 790° C. for 5 minutes (less than the melting point of sodium chloride, which is 804° C.) by means of Spark Plasma Sintering, contemporaneously applying an axial pressure of 40 MPa to the punches. The presence of sodium chloride, the electrical conductivity of which is much smaller than that of the metallic powder, produces a preferential conduction through the metal particles: the electrical discharges among them produce a welding effect, forming the powder into a single and dense body.

To facilitate evacuation of the air and the residual acetone trapped in the interstices internally of the powder, the pressure can be applied after a convenient time or temperature, e.g. after three minutes or after a temperature greater than 300° C. has been reached.

After sintering is complete, the workpiece is cooled to a temperature of below 150° C. and is returned to the normal atmosphere. The acetabular cup 1 is then extracted from the graphite dies and immersed in water up to complete extraction of the sodium chloride granules 12; a slight previous sanding of the porous surface to remove the surface layer can facilitate the extraction of the sodium chloride. With this procedure, an acetabular cup 1 is obtained having a desired geometry, and which is defined by the matrix 8b and the punches 8a and 7: the acetabular cup 1 exhibits an external surface having interconnected porosity of 65% in volume, with the pore dimensions of from 500 to 1000 micron and thickness 1.89 mm. The internal layer of the acetabular cup 1 exhibits a mean density of more than 95%. When extraction is complete, the acetabular component 1 is rinsed carefully to remove sodium chloride residue. The internal surface is then polished or worked with a machine tool for obtaining the final desired finish.

### Example 2

Alumina powder is used, (A16SG, density 3.96 g/cm3), size 0.4 micron.

This powder is mixed with granules 12 of graphite (density 1.84 g/cm3) having dimensions comprised between 250 and 500 microns, with a volume ratio of 40% alumina and 60% graphite. The addition of acetone or another solvent can facilitate homogenisation of the powder 11 with the granules 12 such that the granules 12 are uniformly covered with powder 11. The concave punch 8a, the convex punch 7 and the matrix 8b, made of compact isotropic graphite, are used, with resistance to flexion of more than 65 MPa. The concave punch 8a is inserted in the matrix 8b and filled with the mixture of powder 11 and granules 12, such that the effective volume of material introduced is equal to the final volume desired for the porous layer: for a thickness of the porous layer of 1.89 mm and an external diameter of the acetabular cup 1 of 50 mm, a total effective volume of powder 11 and granules 12 inserted is 4.36 cm³, i.e. a mass of alumina powder of 6.91 g and a mass of graphite of 4.81 g.

The mixture of powder 11 and granules 12, possibly wetted with acetone as required such as to render it more plastic and formable, is distributed in a uniform layer on the concave surface of the concave punch 8a. This operation can be facilitated with use of convex, usually hemispheric, tools. Alternatively, the mixture of powder 11 and granules 12 can be previously and separately formed in the shape of a cup 2 with the use of one or more different forming technologies, e.g. tape casting, injection in hollow dies, moulding, and then inserted in the concave punch 8a after completion of the external forming operations.

The punch 8a and the matrix 8b are loaded with a volume of Alumina powder 11 necessary for obtaining the acetabular cup 1 with a shape and volume desired, following a complete densification of the powder 11 in a Spark Plasma Sintering process.

The convex punch 7 is then inserted in the matrix 8b such that the convex surface of the convex punch 7 goes into contact with the powder to be sintered and formed in the geometry of the punch 7 itself. The surface polishing of the punch will lead to an excellent internal surface finishing of the acetabular cup 1.

The system of punches, matrix and powders is thus placed under vacuum at a pressure of 6 Pa, and heated at a velocity of about 100° C./min up to a sintering temperature selected between 1250° C. and 1550° C., for ten minutes and using Spark Plasma Sintering, contemporaneously applying an axial pressure of 40 Mpa to the punches. The presence of the graphite, which is conductive, while alumina is an electrical insulator, produces a bridge effect across the powder 11 and granule 12 mixture: the electrical discharges between the granules 12 and the heating thereof by the Joule effect produces an effective heating in proximity of the particles of alumina powder 11, facilitating densification and sintering thereof.

To facilitate evacuation of the air and residual acetone trapped in the interstices internally of the powder 11, the pressure can be applied after a convenient time or temperature, for example after 5 minutes or at a temperature higher than 500° C.

After sintering is complete, the workpiece is cooled to a temperature of below 150° C. and is then returned to the normal atmosphere. The acetabular cup 1 is extracted from the forming means and inserted in a controlled-air-flow kiln at a temperature selected from between 800° C. and 1200° C., for eight hours, in order to remove the graphite by means of oxidation. Using this process an acetabular cup 1 is obtained having a desired geometry defined by the matrix 8b and the punches 8a and 7: the acetabular cup 1 exhibits an external surface having interconnected porosity of 60% in volume, with pore sizes comprised between 350 and 700 microns, and with a thickness of 1.89 mm. The internal layer of the acetabular cup 1 exhibits a mean density of more than 94%. The internal surface, alumina having been used, is already sufficiently smooth and polishing to obtain the desired surface finish is not necessary.

The expert in the field can easily use, e.g., compositions such as salts, sulphates, phosphates, nitrates and oxides, in particular of sodium, calcium, potassium, lithium, magnesium, for the granular material 12, using acid or basic solutions as solvents.

Table 1 below reports some examples of granular materials 12 usable and in which way they can be extracted from the acetabular cup (or cotyl) 1 formed.

TABLE 1

| SUBSTANCE IN GRANULES | CONDITIONS OF EXTRACTION |
|---|---|
| Sodium chloride | Distilled water |
| Magnesium Oxide | 6N nitric acid solution; |
| | 6N sulphuric acid solution; |
| | A solution of 60% acetic acid |
| | and 40% distilled water in |
| | volume, maintained at a |
| | temperature of 80° C. < T < 85° C. |
| Calcium oxide | A water solution with a |
| | variable percentage of |
| | methanol, from 0% to 85%; |
| | A solution with chelating |
| | agents such as: |
| | ethylenediaminetetraacetic |
| | acid (EDTA) with alkaline pH; |
| | solutions of diluted acids; |
| Hydroxyapatite | Acidic watery solutions such |
| | as acetic acid, lactic acid; |
| | phosphorus diluted with pH |
| | comprised between 3 and 7; |
| Calcium chloride | Solution with chelating agents |
| | such as: |
| | ethylenediaminetetraacetic |
| | acid (EDTA) with alkaline pH; |
| Graphite | Oxidation in forced air kiln |
| | with a temperature of 800° C. < |
| | T < 1200° C. |

1. A process for realizing biologically-compatible concave and/or convex elements (1), in a system suitable for causing sintering by applying an activating electrical sintering current through a material to be sintered and by applying a pressure on it, the process comprising: sintering in forming means (6), by applying a pressure and at a sintering temperature (T1) a volume (11) of a forming material (11) of an electrically conductive/non-conductive type, thereby obtaining formed concave and/or convex elements (1); and adding to said forming material (11), before said sintering, a granular material (12) which is removable from said formed concave and/or convex elements (1), said granular material (12) having a melting point temperature (T2) which is higher than said sintering temperature (T1),
wherein said granular material (12) comprises:
a granular material having a low level of electrical conductivity, when said forming material (11) is of an electrically-conductive type, whereby the sintering current is concentrated and passes only through the forming material (11), heating it up and sintering it,
or a granular material having a high level of electrical conductivity, when said forming material is of a substantially electrically non-conductive type, whereby the sintering current passes through the granules (12) and heats them up, and the heat so generated is transmitted from the granules to the forming material for sintering it.

2. The process according to claim 1, wherein said forming means (6) comprise pressing means for applying said pressure comprising concave punch means (8a) in which there can be introduced said volume (11) of forming material and said granular material (12), and a convex punch (7) suitable to compress said volume (11) of forming material and granular material (12) in said concave punch (8a).

3. The process according to claim 1, wherein after said sintering said granular material (12) is removed in a removing step from said formed concave and/or convex elements (1), thereby by obtaining concave and/or convex elements (1) having pores (5).

4. The process according to claim 3, wherein said granular material (12) is soluble in a solvent.

5. The process according to claim 3, wherein said removing step comprises dissolving said granular material (12) with a solvent.

6. The process according to claim 1, wherein said granular material (12) exhibits pre-selected granular dimensions.

7. The process according to claim 1, wherein said granular material (12) comprises sodium chloride.

8. The process according to claim 4, wherein said solvent comprises water.

9. The process according to claim 1, wherein said granular material (12) comprises magnesium oxide.

10. The process according to claim 4, wherein said solvent comprises a solvent liquid solution of hydrochloric acid in a quantity comprised between 5 and 50 percent.

11. The process according to claim 10, wherein said solvent liquid solution exhibits a temperature comprised between 0° C. and 90° C.

12. The process according to claim 1, wherein said granular material (12) is selected from among salts, sulphates, phosphates, nitrates and oxides of elements selected from among sodium, calcium, potassium, lithium, magnesium.

13. The process according to claim 1, wherein said adding comprises mixing.

14. The process according to claim 1, wherein said adding comprises applying a layer of said granular material (12) on a surface of a layer of said volume (11) of forming material destined to become a contact surface with a biological surface for gripping to.

15. A biologically-compatible concave and/or convex element (1), obtainable with a process for realizing biologically-compatible elements according to claim 1, wherein it exhibits a concave and/or convex body (2) exhibiting pores (5).

16. The element according to claim 15, wherein said pores (5) are obtained substantially on the surface of said concave and/or convex body (2).

17. The element according to claim 16, wherein said surface comprises a convex surface (4).

18. The element according to claim 15, wherein said pores (5) are obtained on the surface (3, 4) and in the thickness of said concave and/or convex body (2).

19. The element according to claim 15, wherein said concave and/or convex body (2) exhibits a cup-shape.

20. The element according to claim 15, wherein said concave and/or convex body (2) has a cotyl shape.

21. The process according to claim 1, wherein said system suitable for promoting sintering is a Spark Plasma Sintering system.
